(19) United States
(12) Patent Application Publication
(10) Pub. No.: US 2022/0148301 A1
(43) Pub. Date: May 12, 2022

(54) An Auxiliary Diagnostic Model and an Image Processing Method for Detecting Acute Ischemic Stroke

(71) Applicants: West China Hospital of Sichuan University, Chengdu City, Sichuan (CN); University of Electronic science and Technology of China, Chengdu City, Sichuan (CN)

(72) Inventors: Na Hu, Chengdu City, Sichuan (CN); Su Lv, Chengdu City, Sichuan (CN); Shi Gu, Chengdu City, Sichuan (CN); Tianwei Zhang, Chengdu City, Sichuan (CN)

(21) Appl. No.: 17/606,751

(22) PCT Filed: Sep. 29, 2020
(86) PCT No.: PCT/CN2020/118667

§ 371 Date: Oct. 26, 2021

(30) Foreign Application Priority Data
Jun. 10, 2020 (CN) 202010522445.1

(51) Int. Cl. G06V 10/82; G06T 3/40; G06N 3/04

(52) U.S. Cl. CPC G06V 10/82; G06T 3/4046; G06T 2207/20081; G06T 2207/20084; G06N 3/04

(57) ABSTRACT

This invention discloses an auxiliary diagnostic model and an image processing method for detecting acute ischemic stroke. This refers to the technical field of medical image processing. The technical essentials are described as follow: the presented deep-learning model is based on generative adversarial networks (GANs), comprising a generator (G) and a discriminator (D). G is the first three-dimensional convolutional neural network, used to synthesize realistic images from raw data, while D is the second three-dimensional convolutional neural network, used to classify images as real or fake (synthetic). The presented GAN model can learn the mapping relationship from non-enhanced computed tomography (NECT) images to T2-weighted fluid-attenuation inversion recovery (FLAIR) magnetic resonance imaging (MRI), then converting the raw CT to synthetic FLAIR with high sensitivity. This improves the efficiency of emergency scanning in acute ischemic stroke, reaching sensitivity that is poor in CT interpretation and immediacy that is limited in MRI examination.

### TECHNICAL FIELD

This invention refers to the technical field of medical image processing, and discloses an auxiliary diagnostic model and an image processing method for detecting acute ischemic stroke.

### BACKGROUND

Acute ischemic stroke is the most common type of cerebrovascular diseases, and a significant contributor to the global disease burden, bringing a heavy stress and huge consumption to the patients, their families, and the society. Brain assessment of patients with acute ischemic stroke requires both immediacy and sensitivity. In clinical routine, non-enhanced computed tomography (NECT) is the first-line examination in emergency practice. However, it suffers from observer reliability and poor sensitivity to the early small infarction. This easily leads to delayed image interpretation and misdiagnosis, thus affecting timely intervention in the stroke patients.

Magnetic resonance imaging (MRI) holds advantages in detecting small and early cerebral ischemic changes, wherein T2-weighted fluid-attenuation inversion recovery (FLAIR) images demonstrate hyperintensities within 3 to 6 hours after onset of symptoms, but with lower availability, higher expense, and slower image acquisition. This limits MRI to be used in the real emergency settings and degrades it as a supplementary examination for a minority of harsh indications.

Briefly, NECT is rapid but insensitive, whereas MRI is sensitive but time-consuming in early imaging assessment of acute ischemic stroke. Such dilemma has long been existed. Taking advantages of both CT and MRI and integrating them into emergency practices, which balances immediacy with sensitivity, would help improve the diagnostic and therapeutic efficiency of stroke, optimize the stroke management workflow, potentially benefit the patients, their families, and the society. To address the above-mentioned questions, this invention is intended to develop an auxiliary diagnostic model and an image processing method for detecting acute ischemic stroke, especially in emergency practice.

### SUMMARY OF THE INVENTION

The present invention aims to provide an auxiliary diagnostic model and an image processing method for detecting acute ischemic stroke, which builds and trains the generative adversarial network (GAN) model to learn the mapping relationships from NECT to FLAIR images, and then converts the raw CT to synthetic MRI with higher sensitivity. The doctors could search for the suspected patients with these synthetic images rapidly after the head NECT scan. This improves the efficiency of emergency scanning in acute ischemic stroke, reaching both sensitivity that is poor in CT interpretation and immediacy that is limited in MRI examination.

The above mentioned technical purposes of the invention are implemented by the following technical schemes: an auxiliary diagnostic model for detecting acute ischemic stroke, comprising a generative adversarial network model. The generative adversarial network model comprises the first three-dimensional convolutional neural network and the second three-dimensional convolutional neural network. The first three-dimensional convolutional neural network is the generator G that is used to complete 3D image-to-image conversion, and the second three-dimensional convolutional neural networks is the discriminator D that is used to distinguish the authenticity of the input images; the generator G comprises first three-dimensional convolutional layers for downsampling, residual blocks and three-dimensional transposed convolutional layers for upsampling; the discriminator D comprises second three-dimensional convolutional layers and output layers.

By adopting the above-mentioned technical schemes, the auxiliary diagnostic model for detecting stroke is a generative adversarial network model based on two three-dimensional convolutional neural networks. The first three-dimensional convolutional neural networks in the generative adversarial network model is the generator G that is used to complete 3D image-to-image conversion; the second three-dimensional convolutional neural network in the generative adversarial network model is the discriminator D that is used to distinguish the authenticity of the input images; Through the generator G, comprised of two first three-dimensional convolutional layers for downsampling, residual blocks and three-dimensional transposed convolutional layers for upsampling and the discriminator D, comprised of six second three-dimensional convolutional layers and one output layer, the generative adversarial network model can learn the mapping relationships from NECT images to FLAIR images, and using the learned and trained generative adversarial network model, doctors can use the model to generate FLAIR images to assist in the rapid diagnosis of stroke by scanning the brain NECT image during the process of diagnosing stroke, thus improving the efficiency of emergency screening for stroke, and overcoming the clinical predicaments where with the current technology NECT image sensitivity is not high and magnetic resonance images are hard to acquire in time.

The invention is further arranged as follows: the discriminator D adopts a PatchGAN architecture.

By adopting the above-mentioned technical schemes, the PatchGAN architecture is Markov discriminator. By adopting the discriminator D of PatchGAN architecture, the original image input into it has good high resolution and high detail retention.

The invention is further arranged as follows: the generator G comprises two first three-dimensional convolutional layers, six residual blocks and two three-dimensional transposed convolutional layers; the discriminator D comprises six second three-dimensional convolutional layers and one output layer.

By adopting the above-mentioned technical schemes, with the generator G comprised of two first three-dimensional convolutional layers, six residual blocks and two three-dimensional reansposed convolutional layers, it is easy to complete 3D image-to-image conversion.

The invention is further arranged as follows: the network of the generator G uses instance regularization layers and the ReLU layer as the activation function; the network of the discriminator D uses the LeakyRelu layer as the activation function without usage of regularization layers.

By adopting the above-mentioned technical schemes, the network of the generator G uses instance regularization layers and the ReLU layer as the activation function, and the network of the discriminator D uses the LeakyRelu layer as the activation function without usage of regularization layers, then it is easy to ensure the precision of the generative adversarial network model.

An image processing method for acute ischemic stroke, comprising the following steps:

S1, data normalization, collect NECT images of stroke patients and FLAIR images corresponding to NECT images from the hospital, then make data processing of the collected NECT images and FLAIR images, then make data normalization of the collected NECT images and FLAIR images;

S2, model creation, create the generator G to complete 3D image-to-image conversion and the discriminator D to distinguish the authenticity of the input images, and create the generative adversarial network model, the generator G and the discriminator D are two different three-dimensional convolutional neural networks;

S3, model training, define the complete training loss of the generative adversarial network model created in step S2 as

$${G}^{*} = arg {min}_{G} {max}_{D} {L}_{GAN} ( G , D ) + λ {L}_{L 1} ( G ) ,$$

and train the generative adversarial network model, in which, a gradient penalty term is added in the adversarial loss during training processes, and coefficients of the gradient penalty term and L1 loss are both 10;

S4, result generating, after completing the training process for the generative adversarial network model in step S3, the NECT images after the data normalization of stroke patients in step S1 are input into the generator G in the generative adversarial network model, to quickly generate FLAIR images corresponding to NECT images for auxiliary diagnosis.

The invention is further arranged as follows: the generator G in step S2 comprises two first three-dimensional convolutional layers for downsampling, six residual blocks and two three-dimensional transposed convolutional layers for upsampling.

The invention is further arranged as follows: the discriminator D adopts a PatchGAN architecture.

The invention is further arranged as follows: the network of the discriminator D in step S2 uses the LeakyRelu layer as the activation function without usage of regularization layers, and the network of the generator G in step S2 uses the ReLU layer as the activation function with usage of instance regularization layers.

The invention is further arranged as follows: the data normalization in step S1 comprises the following steps:

A, make format conversions on NECT images of stroke patients collected from hospitals and FLAIR images corresponding to NECT images;

B, adopt spm8 clinical toolbox to perform registrations on NECT images and FLAIR images after format conversions in step A, and acquire the registered FLAIR image data and the registered NECT images;

C, make skull stripping operations on the registered FLAIR image data and the registered NECT images in step B, and acquire intracranial FLAIR image data and intracranial NECT image data, then acquire the processed FLAIR image data and the processed NECT image data after the normalization processing on intracranial image data.

The invention is further arranged as follows: a gradient penalty term is added in the adversarial loss of the generative adversarial network model in step S3, and coefficients of the gradient penalty term and L1 loss are both 10; during training process of the model in step S3, when the discriminator D of the generative adversarial network model updates every five times, the generator G updates once.

In summary, the invention has the following beneficial effects: first three-dimensional convolutional neural networks are used as the generator G, to complete 3D image-to-image conversion; second three-dimensional convolutional neural networks are used as the discriminator D, to distinguish the authenticity of images input into the second three-dimensional convolutional neural networks; through the generator G comprised of two first three-dimensional convolutional layers for downsampling, residual blocks and three-dimensional transposed convolutional layers for upsampling and the discriminator D comprised of six second three-dimensional convolutional layers and one output layer, the generative adversarial network model can learn the mapping relationships from NECT images to FLAIR images, and using the learned and trained generative adversarial network model, doctors can use the model to generate FLAIR images to assist in the rapid diagnosis of stroke by scanning the brain NECT image during the process of diagnosing stroke, thus improving the efficiency of emergency screening for stroke, and overcoming the clinical predicaments where with the current technology NECT image sensitivity is not high and magnetic resonance images are hard to acquire in time.

### BRIEF DESCRIPTION OF THE DRAWINGS

FIG. 1 is a structural schematic diagram of the generative adversarial network model in embodiment 1 of the invention;

FIG. 2 is a flowchart of the data standardization in embodiment 2 of the invention;

FIG. 3 is a schematic diagram of the training process of the generative adversarial network model in embodiment 2 of the invention;

FIG. 4 is a schematic diagram of the diagnosis process in embodiment 2 of the invention;

FIG. 5 is a flowchart in embodiment 2 of the invention;

In diagrams, 1, the generative adversarial network model; 2, the generator G; 3, the discriminator D; 4, first three-dimensional convolutional layers; 5, residual blocks; 6, three-dimensional transposed convolutional layers; 7, second three-dimensional convolutional layers; 8, output layers.

### DESCRIPTION OF EMBODIMENTS

Further detailed description of the invention is given below in combination with attached figures from 1 to 5.

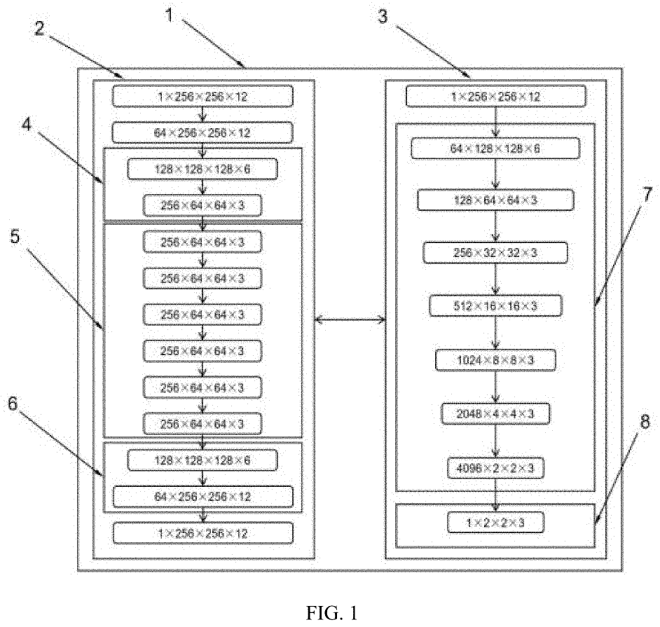

Embodiment 1: An auxiliary diagnostic model for detecting acute ischemic stroke, as shown in FIG. 1, comprising the generative adversarial network model 1, and the generative adversarial network model 1 comprises the first three-dimensional convolutional neural network and the second three-dimensional convolutional neural network, the first three-dimensional convolutional neural network is the generator G2 that is used to complete 3D image-to-image conversion, and the second three-dimensional convolutional neural network is the discriminator D3 that is used to distinguish the authenticity of the input images. The generator G2 comprises two first three-dimensional convolutional layers 4 for downsampling, residual blocks 5 and three-dimensional transposed convolutional layers 6 for upsampling. The discriminator D3 comprises second three-dimensional convolutional layers 7 and output layers 8.

In the embodiment, the auxiliary diagnostic model for detecting stroke is a generative adversarial network model 1 based on two three-dimensional convolutional neural networks. The first three-dimensional convolutional neural network in the generative adversarial network model 1 is the generator G2 that is used to complete 3D image-to-image conversion. the second three-dimensional convolutional neural network in the generative adversarial network model 1 is the discriminator D3 that is used to distinguish the authenticity of the input images. Through the generator G2 comprised of two first three-dimensional convolutional layers 4 for downsampling, residual blocks 5 and three-dimensional transposed convolutional layers 6 for upsampling and the discriminator D3 comprised of second three-dimensional convolutional layers 7 and output layers 8, the generative adversarial network model 1 can learn the mapping relationships from NECT images to FLAIR images, and using the learned and trained generative adversarial network model 1, doctors can use the model to generate FLAIR images to assist in the rapid diagnosis of stroke by scanning the brain NECT image during the process of diagnosing stroke, thus improving the efficiency of emergency screening for stroke, and overcoming the clinical predicaments where with the current technology NECT image sensitivity is not high and magnetic resonance images are hard to acquire in time.

The discriminator D3 adopts a PatchGAN architecture.

In the embodiment, the PatchGAN architecture is Markov discriminator, By adopting the discriminator D of PatchGAN architecture, the original image input into it has good high resolution and high detail retention.

There are two first three-dimensional convolutional layers 4, six residual blocks 5 and two three-dimensional transposed convolutional layers 6.

In the embodiment, with the generator G2 comprised of two first three-dimensional convolutional layers 4, six residual blocks 5 and two three-dimensional transposed convolutional layers 6, it is easy to complete 3D image-to-image conversion.

The network of the generator G uses the ReLU layer as the activation function with usage of instance regularization layers. The network of the discriminator D3 uses the LeakyRelu layer as the activation function without usage of regularization layers.

In the embodiment, the network of the generator G2 uses the ReLU layer as the activation function with usage of instance regularization layers, and the network of the discriminator D3 uses the LeakyRelu layer as the activation function without usage of regularization layers, then it is easy to ensure the precision of the generative adversarial network model.

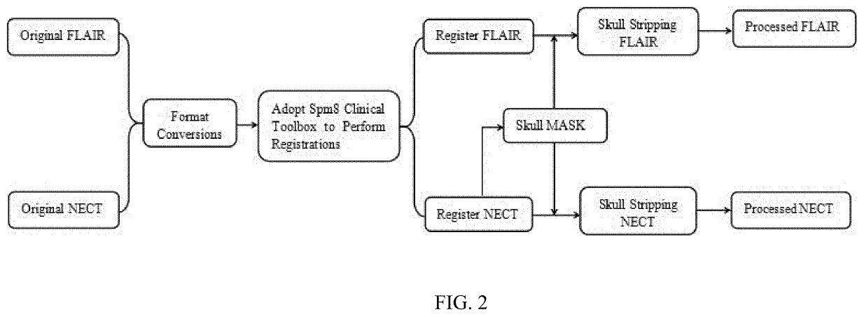

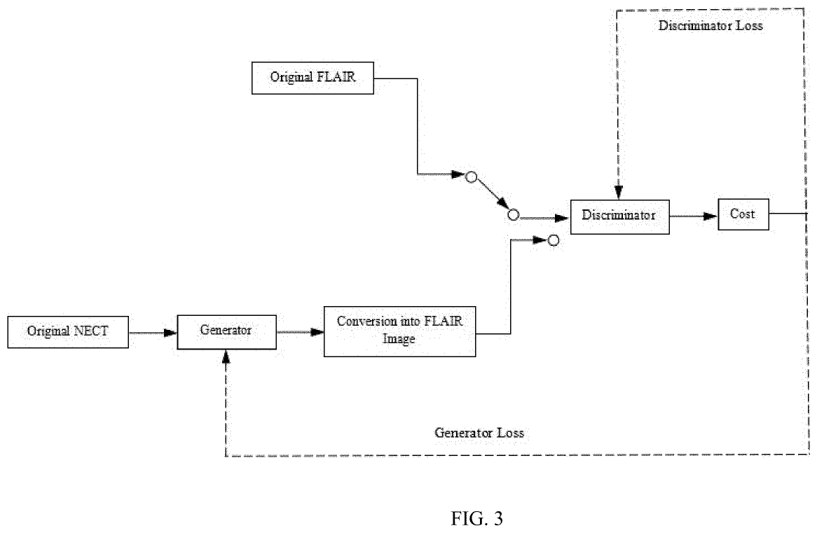

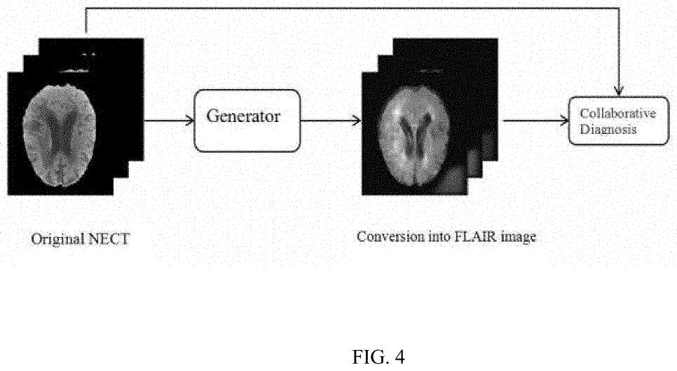

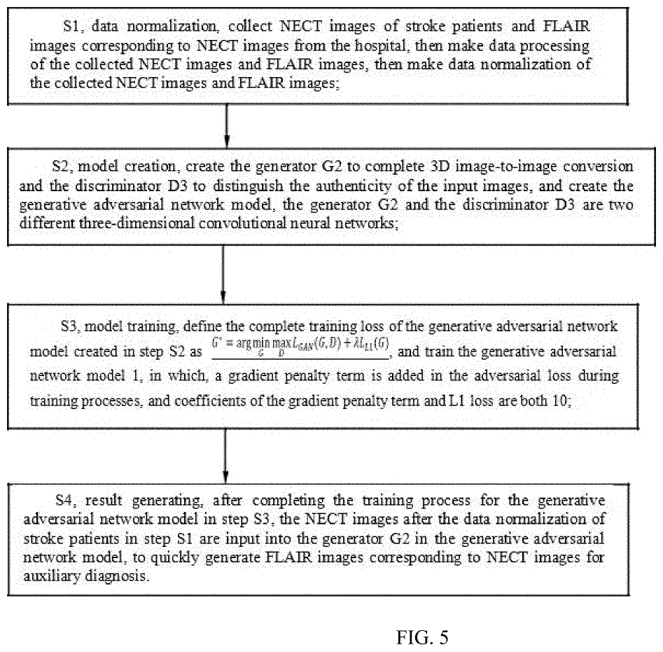

Embodiment 2: an image processing method for detecting acute ischemic stroke, as shown from FIG. 2 to FIG. 5, comprising the following steps:

S1, data normalization, collect NECT images of stroke patients and FLAIR images corresponding to NECT images from the hospital, then make data processing of the collected NECT images and FLAIR images, then make data normalization of the collected NECT images and FLAIR images;

S2, model creation, create the generator G2 to complete 3D image-to-image conversion and the discriminator D3 to distinguish the authenticity of the input images, and create the generative adversarial network model, the generator G2 and the discriminator D3 are two different three-dimensional convolutional neural networks;

S3, model training, define the complete training loss of the generative adversarial network model created in step S2 as

$${G}^{*} = arg {min}_{G} {max}_{D} {L}_{GAN} ( G , D ) + λ {L}_{L 1} ( G ) ,$$

and train the generative adversarial network model 1, in which, a gradient penalty term is added in the adversarial loss during training processes, and coefficients of the gradient penalty term and L1 loss are both 10;

S4, result generating, after completing the training process for the generative adversarial network model in step S3, the NECT images after the data normalization of stroke patients in step S1 are input into the generator G2 in the generative adversarial network model, to quickly generate FLAIR images corresponding to NECT images for auxiliary diagnosis.

The generator G2 in step S2 comprises two first three-dimensional convolutional layers 4 for downsampling, six residual blocks 5 and two three-dimensional transposed convolutional layers 6 for upsampling. The discriminator D3 comprises six second three-dimensional convolutional layers 7 and one output layer 8.

The discriminator D3 adopts a PatchGAN architecture.

The invention is further arranged as follows: the network of the discriminator D in step S2 uses the LeakyRelu layer as the activation function without usage of regularization layers, and the network of the generator G in step S2 uses the ReLU layer as the activation function with usage of instance regularization layers.

The data normalization in step S1 comprises the following steps:

A, make format conversions on NECT images of stroke patients collected from hospitals and FLAIR images corresponding to NECT images;

B, adopt spm8 clinical toolbox to perform registrations on NECT images and FLAIR images after format conversions in step A, and acquire the registered FLAIR image data and the registered NECT images;

C, make skull stripping operations on the registered FLAIR image data and the registered NECT images in step B, and acquire intracranial FLAIR image data and intracranial NECT image data, then acquire the processed FLAIR image data and the processed NECT image data after the normalization processing on intracranial image data.

A gradient penalty term is added in the adversarial loss of the generative adversarial network model in step S3, and coefficients of the gradient penalty term and L1 loss are both 10. During the training process of the model in step S3, when the discriminator D of the generative adversarial network model updates every five times, the generator G updates once.

Working Principle: the auxiliary diagnostic model for detecting stroke is a generative adversarial network model 1 based on two three-dimensional convolutional neural networks. The first three-dimensional convolutional neural network in the generative adversarial network model 1 is the generator G2 that is used to complete 3D image-to-image conversion. the second three-dimensional convolutional neural network in the generative adversarial network model 1 is the discriminator D3 that is used to distinguish the authenticity of the input images. Through the generator G2 comprised of two first three-dimensional convolutional layers 4 for downsampling, residual blocks 5 and three-dimensional transposed convolutional layers 6 for upsampling and the discriminator D3 comprised of second three-dimensional convolutional layers 7 and output layers 8, the generative adversarial network model 1 can learn the mapping relationships from NECT images to FLAIR images, and using the learned and trained generative adversarial network model 1, doctors can use the model to generate FLAIR images to assist in the rapid diagnosis of stroke by scanning the brain NECT image during the process of diagnosing stroke, thus improving the efficiency of emergency screening for stroke, and overcoming the clinical predicaments where with the current technology NECT image sensitivity is not high and magnetic resonance images are hard to acquire in time.

Compared with the traditional method of identifying stroke on NECT in the current study, the ability of using this method to assist in the detection of acute ischemic stroke patients and lesions is significantly improved while reducing time consumption. Sensitivity for emergency personnel such as image technicians and image specialists to use the model and the method to detect stroke patients is 66% to 92%, and the accuracy rate is 67% to 87%, F1 value (a comprehensive index to weigh accuracy rate and precision rate) is 79% to 93%. Compared with the NECT method, sensitivity, accuracy rate and F1 value for the model and the method to detect stroke patients are respectively increased by 159% to 1000%, 124% to 509% and 80% to 618%, and sensitivity, precision rate and F1 value to detect stroke lesions are respectively increased by 278% to 826%, 55% to 134% and 218% to 598%. Meanwhile, speed for emergency staff for detecting patients with acute stroke can be improved by using the model and the method, and time consumption is shortened by 32% to 56% compared with the traditional NECT method.

This specific embodiment is only an explanation of the present invention, which is not a limitation of the present invention. After reading this specification, technicians in the field can make modifications to this embodiment without creative contribution according to requirements, but protected by patent law as long as within the scope of claims of the invention.

1. An auxiliary diagnostic model for detecting acute ischemic stroke, comprising a generative adversarial network model (1), and the generative adversarial network model (1) comprises the first three-dimensional convolutional neural network and the second three-dimensional convolutional neural network; the first three-dimensional convolutional neural network is the generator G (2) that is used to complete 3D image-to-image conversion, and the second three-dimensional convolutional neural networks is the discriminator D (3) that is used to distinguish the authenticity of the input images; the generator G (2) comprises first three-dimensional convolutional layers (4) for downsampling, residual blocks (5) and three-dimensional transposed convolutional layers (6) for upsampling; the discriminator D (3) comprises second three-dimensional convolutional layers (7) and output layers (8).

2. The auxiliary diagnostic model for detecting acute ischemic stroke according to claim 1, wherein the discriminator D (3) adopts a PatchGAN architecture.

3. The auxiliary diagnostic model for detecting acute ischemic stroke according to claim 1, wherein the generator G (2) comprises two three-dimensional convolutional layers (4), six residual blocks (5) and two three-dimensional transposed convolutional layers (6).

4. The auxiliary diagnostic model for detecting acute ischemic stroke according to claim 1, wherein the network of generator G (2) uses the ReLU activation function with usage of the instance regularization layer; the network of discriminator D (3) uses the LeakyRelu activation function without usage of the regularization layer.

5. An image processing method for detecting acute ischemic stroke, comprising the following steps:
S1, data normalization, collect NECT images of stroke patients and FLAIR images corresponding to NECT images from the hospital, then make data processing of the collected NECT images and FLAIR images, and then make data normalization of the collected NECT images and FLAIR images;
S2, model creation, create the generator G (2) to complete 3D image-to-image conversion and the discriminator D (3) to distinguish the authenticity of the input images, and create the generative adversarial network model (1), the generator G (2) and the discriminator D (3) are two different three-dimensional convolutional neural networks;
S3, model training, define the complete training loss of the generative adversarial network model (1) created in step S2 as
$${G}^{*} = arg {min}_{G} {max}_{D} {L}_{GAN} ( G , D ) + λ {L}_{L 1} ( G ) ,$$
and train the generative adversarial network model (1), in which, a gradient penalty term is added in the adversarial loss during training processes, and coefficients of the gradient penalty term and L1 loss are both 10;
S4, result generating, after completing the training process for the generative adversarial network model (1) in step S3, the NECT images after the data normalization of stroke patients in step S1 are input into the generator G (2) in the generative adversarial network model (1), to quickly generate FLAIR images corresponding to NECT images for auxiliary diagnosis.

6. The image processing method for detecting acute ischemic stroke according to claim 5, wherein the generator G (2) in step S2 comprises two first three-dimensional convolutional layers (4) for downsampling, six residual blocks (5) and two three-dimensional transposed convolutional layers (6) for upsampling; the discriminator D (3) comprises six second three-dimensional convolutional layers (7) and one output layer (8).

7. The image processing method for detecting acute ischemic stroke according to claim 5, wherein the discriminator D (3) adopts a PatchGAN architecture.

8. The image processing method for detecting acute ischemic stroke according to claim 5, wherein the network of the discriminator D (3) in step S2 uses LeakyRelu as the activation function without usage of regularization layers, and network of the generator G (2) in step S2 uses ReLU as the activation function with usage of instance regularization layers.

9. The image processing method for detecting acute ischemic stroke according to claim 5, wherein the data normalization in step S1 comprises the following steps:
A, make format conversions on NECT images of stroke patients collected from hospitals and FLAIR images corresponding to NECT images;
B, adopt spm8 clinical toolbox to perform registrations on NECT images and FLAIR images after format conversions in step A, and acquire the registered FLAIR image data and the registered NECT images;
C, make skull stripping operations on the registered FLAIR image data and the registered NECT images in step B, and acquire intracranial FLAIR image data and intracranial NECT image data, then acquire the processed FLAIR image data and the processed NECT image data after the normalization processing on intracranial image data.

10. The image processing method for detecting acute ischemic stroke according to claim 5, wherein the gradient penalty term is added in the adversarial loss of the generative adversarial network model (1) in step S3, and coefficients of the gradient penalty term and L1 loss are both 10; during processes of the model training in step S3, when the discriminator D (3) of the generative adversarial network model (1) updates every five times, the generator G (2) updates once.
